(19) United States
(12) Patent Application Publication
(10) Pub. No.: US 2007/0059778 A1
(43) Pub. Date: Mar. 15, 2007

(54) Modified colony assay

(76) Inventors: Albertus Wernerus Wognum, Vancouver (CA); Sharon A. Louis, North Vancouver (CA); Gary Peter Dossantos, Vancouver (CA); Terry E. Thomas, Vancouver (CA)

Correspondence Address: BERESKIN AND PARR, 40 KING STREET WEST, BOX 401, TORONTO, ON, M5H 3Y2, CA

(21) Appl. No.: 11/518,953

(22) Filed: Sep. 12, 2006

Related U.S. Application Data
(60) Provisional application No. 60/715,579, filed on Sep. 12, 2005.

(51) Int. Cl. G01N 33/567

(52) U.S. Cl. 435/7.2

(57) ABSTRACT

Improved colony forming cell (CFC) assays are described. The improved assay comprises modifications to the standard CFC assay that enable analysis of temporal, real-time changes in antigen expression during colony development without need to fixate or destroy the culture. The improved assay is applicable to hematopoietic CFC assays as well as to CFC assays for other cells, such as neural cells and mammary cells. In one embodiment, the invention comprises adding a detection reagent, most likely a fluorescently labeled antibody, that is specific for antigens (most likely a cell-surface antigen) expressed on progenitors or on specific mature cell types to a culture at the start or during culture. The invention also comprises modifications to the culture medium and cell preparations used in standard CFC assays to selectively promote the development of one colony type while preventing or suppressing the development of other colony types.

This application claims the benefit under 35 U.S.C.§119(e) of U.S. provisional patent application No. 60/715,579 filed Sep. 12, 2005, which is incorporated herein by reference in its entirety.

### BACKGROUND OF THE INVENTION

Hematopoietic CFC-assays

In the adult, hematopoiesis is maintained by the constant regulated production of billions of mature blood cells derived from a small population of relatively quiescent hematopoietic stem cells (HSC) located in the bone marrow. These cells have the potential to undergo self-renewal to maintain their numbers and to produce cells of all the hematopoietic lineages. Proliferation and differentiation of HSC produces a heterogeneous compartment of actively dividing cells called hematopoietic progenitors. With progressive cell divisions, these progenitors have limited self-renewal capacity and become committed to the various blood cell lineages i.e. white blood cells, red blood cells and platelets.

Since their introduction more than 30 years ago, colony assays have been used extensively for research and clinical applications of hematopoietic cells in particular. These include identification of stimulatory and inhibitory growth factors, supportive diagnostic assays of myeloproliferative disorders and leukemias, and evaluation of the hematopoietic proliferative potential of bone marrow (BM), cord blood (CB) and mobilised peripheral blood (MPB) samples for clinical transplantation. Hematopoietic progenitor cells will proliferate and differentiate in vitro to produce distinct colonies in semi-solid media. Culture conditions that promote such colonies have been optimised and standardised offering a reproducible functional assay for hematopoietic progenitors. This assay has been called the colony forming cell (CFC) assay. The CFC assay has become the benchmark functional assay to assess the ability of various hematopoietic cell sources to divide and differentiate, especially following ex vivo manipulations including T-cell depletion, CD34⁺ cell enrichment, gene therapy protocols and cryopreservation. The CFC assay is also used to screen novel drug candidates for potential toxicity on hematopoietic cells.

Hematopoietic CFC assays are used to quantify multi-potential progenitors and single lineage restricted progenitors of the erythroid, granulocytic, monocyte-macrophage and megakaryocytic pathways. The most commonly used procedure involves the plating of a single cell suspension into semi-solid nutrient medium supplemented with the appropriate combinations of cytokines that supports the proliferation and differentiation of individual progenitor cells into discrete colonies containing recognizable progeny. The CFC are classified and enumerated based on morphologic recognition of mature cells within the colony in situ by light microscopy. The number of colonies obtained should be linearly proportional to CFC content of the input cell suspension provided that a sufficiently low number of cells are plated. In general, under ‘optimal’ assay conditions, colonies containing cells of two or more lineages (mixed colonies) arise from a more primitive progenitor than those containing cells of a single lineage. In addition, more immature progenitors generate larger colonies and require a longer period of time in culture to allow maturation of the cells within the colony.

The various types of colonies are described below. Erythroid progenitors [colony-forming unit-erythroid (CFU-E) and burst-forming unit-erythroid (BFU-E)] are detected by their ability to produce colonies of maturing erythroblasts in the presence of erythropoietin (EPO). The distinctive reddish color of the hemoglobin in erythroblasts of mature CFU-E and BFU-E colonies assists in their recognition. Myeloid progenitors [CFU-granulocyte (CFU-G), CFU-macrophage (CFU-M), and CFU-GM] produce colonies that contain 50 to several thousand granulocytes, macrophages or both cell types in the presence of one or more of the following cytokines- GM-colony stimulating factor (GM-CSF), Interleukin-3 (IL-3), IL-6, G-CSF, and Stem Cell Factor (SCF). Because of their primitive nature, CFU-GEMM tends to produce large colonies of >500 cells containing erythroblasts and recognizable cells of at least one other lineage. The use of an optimal combination of cytokines, that supports the differentiation of multiple lineages, allows the detection of CFU-E, BFU-E, CFU-GM and CFU-GEMM within the same culture. The most widely used media formulations contains 0.8-1.0% methylcellulose as the semi-solid matrix, fetal bovine serum (FBS), bovine serum albumin (BSA) and recombinant cytokines. Less widely used are CFC assays that contain agar or collagen as the semisolid matrix.

Colony enumeration is performed using a quality inverted microscope for detection of colonies in methylcellulose-based cultures. An inverted microscope equipped with 10 or 12.5× eyepiece objectives and planar objectives 2×, 4× and 10× is recommended. The colonies are identified based on size and morphological characteristics. CFU-E-derived colonies are best detected using a magnification of 40-50×. BFU-E, CFU-GM and CFU-GEMM can then be scored at the lower magnification and a higher magnification can be used to confirm colony type.

Colony identification on the basis of size and morphology is inherently subjective and there can be considerable variation in the determination of absolute CFC (Bacigalupo et al. Bone Marrow Transplantation (1995); Torres et al. Blut (1985)). Inconsistent scoring can result from differences in the criteria for colony identification between groups and inexperience of laboratory personnel (Burger et al., Transfusion (1999); Lumley et al. Bone Marrow Transplantation (1999); Lamana et al., Bone Marrow Transplantation (1999)). When training new personnel, comparative counting with experienced staff provides the most useful form of instruction and should be continued until an adequate level of proficiency is attained. Even for accomplished staff, cross-comparisons once a month of counts with common set of progenitor cultures is recommended to maintain reproducible scoring. In addition, different types of colonies can only be counted at the end of the culture period when colonies have sufficiently matured. Individual colonies can be identified at earlier time points during culture, but distinction between colony types on the basis of differences in morphology or degree of hemoglobinization is not possible as the cells have not been sufficiently matured to be morphologically distinguishable. Therefore, existing CFC assay formats cannot be used to obtain information about the development of different lineages at various time points during the course of the cultures.

Hematopoietic cells representing different stages of differentiation and different lineages display many different proteins and other molecules. The types and amount of these molecules is characteristic for the differentiation stage and lineage and detection of these molecules provides approaches to identify the different cell types in blood, bone marrow and other hematopoietic tissues, in purified cell preparations and in cultures of hematopoietic cells. HSCs and progenitors express molecules, e.g., CD34, that are absent or only weakly expressed on mature blood cells. Mature red blood cells and their immediate precursor cells express Glycophorin-A (GpA), but are negative for, or only weakly express, another molecule, CD45. On the other hand most white blood cells (which include lymphocytes, granulocytes and monocyte/macrophages) do not express GpA, but are positive for CD45 and for other molecules that define specific white blood cell subsets, for example Ly-6G/-C for mouse granulocytes. Mature platelets and their precursors, megakaryocytes, express CD41 which is absent from all other lineages. Detection of the expression of these various molecules on hematopoietic cells can be done using specific antibodies and various detection methods. Such approaches can in principle be used to identify different colony types in hematopoietic CFC assays, but available detection methods, e.g., flow cytometry, immunofluorescence microscopy and immunocytochemistry, can only be performed at the end of the culture period as they involve harvesting and breaking up of individual colonies (for flow cytometry), or require drying and chemical fixation of the whole cultures before staining colonies in situ. Drying down and staining of colonies can be performed for CFC assays done in collagen or agar-based media, but it is technically difficult to preserve colony morphology and prevent nonspecific staining. This approach cannot be performed in the majority of hematopoietic CFC assays, which are done in methylcellulose-based medium, since colony morphology is completely destroyed.

Modifications to the detection methods that improve the distinction between different colony types at the end of the culture as well as monitoring the development of individual colony types during culture would facilitate the use of the CFC assay for research and clinical applications. For example, it would make CFC assays faster, more accurate and more reproducible. It would also make it possible to investigate possible stage and lineage-specific effects of cytokines, drugs and other compounds on hematopoietic cell development.

The accuracy and usefulness of the CFC assay is also determined by the composition of the culture medium and of the cell preparation. Culture media in common usage for erythroid and myeloid CFC assays consist of 0.8-1.0% methylcellulose in Iscove's MDM, Fetal Bovine Serum (FBS), Bovine Serum Albumin (BSA) and recombinant cytokines (with phenol red for color). Identification of colonies could become faster and more accurate if only development of only a single colony type is supported in a CFC assay, while the development of other colony types is suppressed. The combination and concentration of cytokines determine the type and size of colonies as well as the efficiency with which the colonies develop. For example, erythropoietin (EPO) is critical for full development of erythroid development of normal erythroid progenitors, but by itself it only stimulates relatively mature erythroid progenitors, called CFU-E, which form small colonies containing 8-16 hemoglobinized cells. In combination with other growth factors, SCF in particular, that act on more primitive hematopoietic cells, EPO stimulates development of more primitive erythroid progenitors, called BFU-E that form large clusters of hemoglobinized cells. Similarly, development of myeloid colonies require combinations of cytokines, e.g., SCF, IL-3, IL-6, GM-CSF and G-CSF, which act on primitive cells as well as on differentiating granulocyte and monocyte/macrophage progenitor cells.

One problem with the standard CFC assay is background CFC development due to cytokines and other compound in the medium. This can be overcome by using serum free media that contain defined, purified components.

Another problem with the standard CFC assay is background CFC formation due to cytokines produced by accessory cells present in the cell preparation. Accessory cells are cells that do not produce colonies themselves but that support CFC formation by other cells by supplying essential nutrients, cytokines or other compounds. Such accessory function can be provided by mature cells or nonhematopoietic cells present in the cell suspension. The undesired CFC formation due to accessory cells could be eliminated or suppressed by modifying the composition of the cell preparation, e.g., removing accessory cells selectively or by purifying hematopoietic CFCs.

Eliminating background erythroid colony formation by normal progenitors is important for diagnostics and research of myeloproliferative disorders in which a proportion of the pathologic progenitors are cytokine, (in particular EPO-) independent.

Recently, CFC assays have also been developed for non-hematopoietic cell types, e.g., neural and mammary cells.

Neural CFC Assay

Neural stem cells (NSCs) were initially identified in liquid suspension cultures by their ability to proliferate into neurospheres and to form a large number of progeny of the three neural cell lineages, i.e., astrocytes, neurons and oligodendrocytes, while exhibiting self-renewal and retaining their multilineage potential over time (Reynolds et al., J. Neurosc. (1992)). The neurosphere assay does not distinguish directly between neural stem cells and more mature progenitors. As neurospheres are cultured in suspension and can move freely through the medium they may break up or aggregate during culture. For these reasons, the neurosphere assay may not provide reliable information on clonal growth properties and frequencies of neural stem cells and progenitors. The neural CFC assay is a semisolid collagen-based culture system that supports clonal growth of neural stem cells and progenitors, and allows their quantitation and distinction between neural stem cells and more differentiated cells. Neural stem cells and progenitors are distinguished on the basis of their proliferative response. Cells with the highest proliferative potential form the largest colonies (>2 mm diameter) and fulfill all the functional criteria for the NSCs, i.e., capacity for self renewal and differentiation into neurons, astrocytes and oligodendrocytes. Cells with low proliferative capacity form small colonies (<2 mm diameter) and represent progenitors with limited proliferation and differentiation abilities (Reynolds et al. 2003 Abstract Viewer/Itinerary Planner, Washington D.C., Society for Neuroscience, 2003).

Neural stem cells and differentiated cells of the various neural cell lineages can be identified by staining with specific antibodies that recognize specific antigens expressed on these cells. For the purpose of this invention markers that are expressed on the cell-surface are of particular interest, as these markers can be detected on intact viable cells without need to fixate or permeabilize the cells. Several markers have been identified that are expressed on the cell-surface of stem cells and progenitors but absent form mature cells. These include, for example, CD133, CD56 (NCAM) and CD15 (Singh et al., Cancer Research 63: 5821-5828, 2003; Capela and Temple; Dev. Biol. 291: 300-313, 2006). Examples of markers for mature neural cells include the astrocyte marker Glial Fibrillary Acidic Protein (GFAP), the oligodendrocyte markers Myelin Basic Protein (MBP) and the neural markers Neurofilaments NF-H, NF-L and NFL-M as well as Neuronal Class III beta-Tubulin. These markers can be used to distinguish stem cells and mature cells in adult and embryonic neural tissues, but also to identify brain tumour stem cells and their differentiated progeny in primary and cultured cells of various human brain tumours, including for example, glioblastoma multiforme.

Mammary CFC Assay

In vitro analysis of human mammary CFCs enables the distinction of three distinct progenitors within human mammary epithelium: The luminal-restricted progenitor (CFC-Lu), the myo-epithelial-restricted progenitor (CFC-Me) ant the bipotent progenitor (CFC-LuMe), a progenitor that can generate both luminal and myoepithelial cells (Pechoux et al. (1999)). Similar CFCs have been identified in the mouse mammary gland (Smalley (1998, 1999)). Myo-epithelial and luminal cells and their precursors express various cell surface markers that can be used to identify and distinguish these cells. Markers preferentially expressed on myoepithelial cells and their precursors include CD24, Keratin-18, Keratin-19, EpCAM and MUC-1 as well as the antigen recognized by the monoclonal antibody clone 5E11 (StemCell Technologies cat #01422). Another marker, CD10, is preferentially expressed on myo-epithelial cells and progenitors, while primitive mammary cells, including mammary stem cells can be identified by combinations of markers, such as CD24 and CD49f.

### SUMMARY OF THE INVENTION

This invention comprises modifications to the standard CFC assay that enable analysis of temporal, real-time changes in antigen expression during colony development without need to fixate or destroy the culture. The improved assay is applicable to hematopoietic CFC assays as well as to CFC assays for other cells, such as neural cells and mammary cells.

In one aspect, the invention comprises adding a detection reagent, preferably a fluorescently labeled antibody, that is specific for antigens (preferably a cell-surface antigen) expressed on progenitors or on specific mature cell types to a culture at the start or during culture. Examples of antigens include GPA for erythroid cells, CD45 for white blood cells and CD34 for progenitors. The labeled antibody is present throughout subsequent culture, and binds to colonies while they develop and express the antigen for which the antibody is specific. By periodically inspecting the colonies using an inverted fluorescence microscope, specific colony types can be identified as they start expressing lineage-specific antigens, bind the labeled antibody and become fluorescent. Other colonies, that do not express the antigen remain non fluorescent, while colonies that lose expression of certain antigens during development may be fluorescent at early time points, but lose fluorescence later. The invention thus enables the researcher to distinguish colonies on the basis of antigen expression as well as size and morphology. This facilitates accurate and rapid identification and enumeration of specific colony types at the end of culture, and also enables the researcher to follow the kinetics of the development of different colony types during culture on the basis of changes in antigen expression. This is an improvement over the standard CFC assay method of detecting colonies which uses size and morphology, which is inherently subjective and wherein certain colonies can only be detected at the end of the culture period.

Accordingly, in one aspect, the present invention provides a method of detecting progenitor cells in a starting cell preparation comprising:

(a) culturing the cell preparation in a culture medium suitable to promote the growth and differentiation of the progenitor cells into colonies containing a specific cell type;

(b) adding a detection reagent that can detect the progenitor cells or the specific cell type; and

(c) detecting progenitor cells or the specific cell type wherein the presence of such cells indicates the presence of progenitor cells in the starting cell preparation.

The invention also comprises modifications to the culture medium and cell preparations used in standard CFC assays to selectively promote the development of one colony type while preventing or suppressing the development of other colony types. In one embodiment, the development of erythroid colonies is promoted and the development of myeloid colonies inhibited by use of serum free medium supplemented with the cytokines SCF and EPO. The absence of serum prevents background colony formation caused by cytokines or other stimulatory compounds present in fetal bovine sera used commonly in culture assays. In another embodiment, background colony formation in the absence of cytokines is prevented or inhibited by using modified cell preparations from which accessory cells, which do not form colonies themselves but can support the growth of specific colony types, are removed by depleting specific cell types, e.g., monocytes, or by enriching progenitor cells.

Accordingly, in another aspect, the present invention provides a method of detecting progenitor cells to a specific cell type in a starting cell preparation comprising:

(a) depleting a population of cells that are not the specific cell type in the cell preparation;

(b) culturing the depleted cell population in a culture medium suitable to promote the growth and differentiation of the progenitor cells into colonies containing the specific cell type; and

(c) detecting the progenitor cells or the specific cell type in the cell population.

In one embodiment, the starting cell preparation is depleted of monocytes prior to culture. Such an embodiment may be used to detect erythroid progenitors. Monocytes may be depleted using techniques known in the art including a negative selection method using antibodies that bind to CD14⁺ monocytes. In such an embodiment, the culture medium can be serum-free medium supplemented with SCF. In a specific embodiment, the culture medium does not contain EPO.

The various configurations of the invention can be combined to provide more accurate and reproducible CFC assays for specific colony types, which have applications in stem cell research, drug discovery toxicity screening and diagnosis of myeloproliferative disorders in particular.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1: Staining of erythroid colonies in a hematopoietic CFC assay. Bright field image (A, C) and fluorescence image of a section of dishes stained with the negative control (A,B) or anti-GpA-FITC (C,D). Original magnification: 65×.

FIG. 2: Staining of erythroid colonies in a hematopoietic CFC assay. Composite fluorescence image of a whole dish stained with anti-GPA-FITC. Original magnification: 65×.

FIG. 3: Fluorescence intensity of an erythroid colony stained with anti-GPA-FITC at the start of the culture and measured after 7, 11 and 14 days of culture.

FIG. 4: Effect of 5-Fluoro-Uracil (5-FU) on colony size (A) and fluorescence intensity (B) of erythroid colonies stained with anti-GPA-FITC. Results are the average for 9 colonies cultured without exposure 5-FU and 5 colonies cultured with 5-FU.

FIG. 5: Staining of CD45⁺ colonies in a hematopoietic CFC assay. Bright field images (A, C) and fluorescence image (B, D) of a section of dishes stained with the negative control (A,B) or anti-CD45-FITC (C,D). Original magnification: 65×.

FIG. 6: Staining of granulocyte colonies in a mouse hematopoietic CFC assay. Bright field image (A) and fluorescent image (B) of a section of a dish stained with a rat anti-mouse-Gr-1-FITC antibody conjugate. The arrows indicate granulocyte colonies, which are brightly stained with the anti-Gr-1-FITC. Original magnification: 162.5×.

FIG. 7: Staining of human brain tumour colonies in a human NCFC assay. Bright field image (A,C) and fluorescent image (B,D) of a section of a dish stained with an anti-CD15-FITC antibody conjugate (A,B) and a control antibody (anti-KLH-FITC) (C,D). The arrow indicates a NCFC colony, which is brightly stained with the anti-CD15-FITC. Original magnification: 65×.

### DETAILED DESCRIPTION OF THE INVENTION

As hereinbefore mentioned, the invention relates to improved CFC assays. In one aspect, the improvement comprises adding a detection reagent that facilitates accurate and rapid identification of different colony types during culture.

Accordingly, the present invention provides a method of detecting progenitor cells in a starting cell preparation comprising:

(a) culturing the cell preparation in a culture medium suitable to promote the growth and differentiation of the progenitor cells into colonies containing a specific cell type;

(b) adding a detection reagent that can detect the progenitor cells or the specific cell type; and

(c) detecting progenitor cells or the specific cell type wherein the presence of such cells indicates the presence of progenitor cells in the starting cell preparation.

The detection reagent can be any reagent that can detect or identify progenitor cells or specific cell types. Progenitor cells or specific cell types are generally detected using a CFC assay wherein progenitor cells in the starting cell preparation differentiate into discrete colonies containing recognizable cells of a specific cell type. Preferable, the detection reagents are antibodies that are specific for cell types present in one colony type, but not in other colony types. Preferably these antibodies are labeled with fluorescent molecules, e.g., fluorescein, thus enabling detection of specific colony types by fluorescence microscopy without interference by other colony types. This increases the ability to accurately identify and enumerate specific colony types.

A range of antibody conjugates are available to stain specific colony types. These can be added to the cultures at the end of the culture period and bind to antigens expressed on the surface of intact cells. Alternatively, these can be added to the culture at the beginning of the culture period to enable binding to colonies during their development thus enabling analysis of changes in antigen expression during development of specific colony types.

Examples of Antibody Conjugates:

1. Antibodies against antigens expressed specifically on erythroid cells, e.g., Glycophorin A, and conjugated with fluorochromes, such as fluorescein, can be used to selectively stain colonies of erythroid cells and distinguish these from myeloid and other colony types.

2. Antibodies against antigens expressed on macrophages and granulocytes, e.g., CD11b, and conjugated with fluorochromes such as fluorescein, can be used to selectively stain and detect colonies containing these cells and distinguish these from erythroid and other colony types. Similarly, conjugated antibodies against CD45 can be used to stain myeloid and lymphoid colonies, as well as colonies that contain immature cells, which are CD45⁺, and distinguish these colonies from non-hematopoietic and mature erythroid colonies, which are CD45⁻.

3. The use of antibody conjugates is not limited to the detection and distinction of erythroid and myeloid colonies. By choosing appropriate antibodies, e.g., anti-CD19 or anti-CD20 for B cells, anti-CD3 for T cells, colonies consisting of other cell types can be detected as well. Individual cultures can also be stained simultaneously with two or more antibodies that have been conjugated with different fluorochromes (e.g., Fluorescein and Texas Red) to detect different colony types in the same dish or to detect different cell types in the same colony. Examples of the latter are monocytes, which can be selectively stained with antibodies against CD14, and granulocytes, which can be selectively stained with antibodies against CD15 or CD66b, in colonies that are derived from CFU-GM progenitor cells and that contain both cell types.

4. Antibodies against astrocytes, oligodendrocytes and neurons can be used to distinguish colonies containing these cell different neural types in neural CFC assays and to monitor changes the development of colonies containing these cell types during culture. Astrocyte markers expressed on the cell surface include Glial Fibrillary Acidic Protein (GFAP), oligodendrocyte markers include Myelin Basic Protein (MBP) and neural markers include Neurofilaments NF-H, NF-L and NFL-M as well as Neuronal Clas III beta-Tubulin. Neural colonies consisting of, or containing primitive cells can be identified by supplementing the cultures with labeled antibodies against markers, such as NCAM (CD56) and CD15, which are preferentially expressed on primitive cells. Labelled antibodies against other markers, for example the Nerve Growth Factor Receptor CD271, can be used to identify neural colonies, irrespective of their lineage, while non-antibody staining reagents, such as FITC-conjugated lectins, can be used for the same purpose as well.

5. Antibodies against mammary stem cells, myo-epithelial cells or luminal epithelial cells can be used to detect colonies containing these cells in mammary CFC-assays. Markers for luminal epithelial cells and/or progenitors include CD24, the marker recognized by the monoclonal antibody clone 5E11 (StemCell Technologies cat #01422), Keratin-18, Keratin-19, EpCAM and MUC-1. Markers for myo-epithelial cells and progenitors include CD10. Colonies that consist of, or contain, mammary stem cells can be detected using combinations of antibodies against markers that are preferentially co-expressed on primitive cells, e.g., CD24 and CD49f.

The antibody conjugates can be prepared using techniques known in the art including the preparation of covalent antibody conjugates prepared by chemical conjugation as well as tetrameric antibody conjugates. Bispecific tetrameric antibody conjugates (TACs) comprising mouse IgG1 antibodies against markers expressed on specific colonies and antibodies against a fluorochrome, such as fluorescein or phycoerythrin, and cross-linked together by a monoclonal rat antibody against mouse IgG1 can be used as an alternative to covalent antibody conjugates. TACs are stable at 37° C. and can be added to the culture at the beginning of the culture period without loss of their ability to bind to specific antigens.

By combining different fluorescent antibody conjugates it is possible to detect different colony types in the same culture. It is also possible to identify multilineage colony types that contain several cell types and that are very difficult to detect on the basis of size and morphology alone.

The starting cell preparation can be any cell preparation comprising progenitor cells, including but not limited to hematopoietic tissues, such as blood, bone marrow, cord blood, placenta, spleen, fetal liver, yolk sac, aorta-gonad-mesonephros region, or nonhematopoietic tissues that contain stem cells, such as adult liver, heart, skeletal muscle, CNS tissue, mammary tissue, adipose tissues, and also cultured stem cells. The cells or tissue can be from any source including embryonic, post-natal or adult tissue from a mammal such as a human or rodent. The cells may be obtained from normal or tumor tissue.

The cell preparation is cultured in a culture medium and for a period of time that is suitable for the growth and differentiation of the progenitor cells. Preferably the culture medium is a semi-solid medium such as a methylcellulose, agar, collagen or plasma clot based culture medium. Preferably, the culture medium is methylcellulose based.

In one embodiment, the culture medium is modified to comprise components to selectively support the growth of colonies of a specific type and exclude or inhibit the growth of colonies of other types. Standard colony assays comprise components that support erythroid, myeloid and multipotent colonies, necessitating the need to distinguish different colony types in the same culture. Culture media formulations can be prepared that support the growth of only a single colony type and thus reduce the variability and time required for identification and enumeration of these colonies. One such culture medium comprises combinations of factors that selectively promote proliferation of erythroid colonies. Such combination of factors would include Stem Cell Factor (SCF) and erythropoietin (EPO, but excludes factors such as Interleukin-3 (GM-CSF) and Granulocyte/Macrophage Colony-Stimulating Factor (GM-CSF), which support CFU-GM colonies. Another culture medium comprises factors that prevent formation of erythroid colony growth from normal CFCs, but supports factor-independent erythroid colony growth from patients with hematopoietic malignancies. Such factors include SCF, but exclude EPO. Such modified culture medium also excludes serum, which contains EPO and other factors that can support normal erythroid colonies, and thus could cause background colony formation, but could include neutralizing antibodies against EPO and other factors present in the culture media or produced by cells in the cell preparation and render these inactive. In one application such modified culture media is used in combination with a modified culture system with cell preparations from which CFCs are purified or mature cells and non-CFCs are depleted to reduce background colony formation.

In another embodiment, the culture system would reduce the variability in the types of different colony types by modifying the preparation of the cell suspension itself. This can be achieved by purifying colony forming cells, by removing mature cells and non-colony-forming cells that produce factors that can affect the growth of colony-forming cells. Colony-forming cells can be purified by staining cell preparations with antibodies or antibody conjugates that are specific against antigens, such as CD34, CD117 and CD133, which are selectively expressed on the surface of CFCs and other immature hematopoietic cells, but absent from most mature cells and non-CFCs. Mature cells and non-CFCs can be depleted by staining cell preparations with antibodies or antibody conjugates that are specific against antigens that are absent from mature cells and non-CFC. Examples of such antigens are CD2, CD3, CD11b, CD11c, CD14, CD16, CD19, CD56, CD66b and Glycophorin-A. A variety of methods can be used to separate the antibody stained cells from non-stained cells. Examples include fluorescence-activated cell sorting (e.g., FACS™, BD-Biosciences. San Jose, Calif.) immunomagnetic Separation methods such as MACS™, (Miltenyi Biotec, Bergisch Gladbach, Germany), EasySep® and StemSep®, (StemCell Technologies, Vancouver, Canada) and density-centrifugation methods such as RosetteSep™ and SpinSep™ (StemCell Technologies).

Accordingly, in another aspect, the present invention provides a method of detecting progenitor cells to a specific cell type in a starting cell preparation comprising:

(a) depleting a population of cells that are not the specific cell type in the cell preparation;

(b) culturing the depleted cell population in a culture medium suitable to promote the growth and differentiation of the progenitor cells into colonies containing the specific cell type; and

(c) detecting the progenitor cells or the specific cell type in the cell population.

In one embodiment, the starting cell preparation is depleted of monocytes prior to culture. Such an embodiment may be used to detect erythroid progenitors. Monocytes may be depleted using techniques known in the art including a negative selection method using antibodies that bind to CD14⁺ monocytes. In such an embodiment, the culture medium can be serum-free medium supplemented with SCF. In a specific embodiment, the culture medium does not contain EPO.

### EXAMPLES

The following non-limiting examples are illustrative of the present invention:

### Example 1

### Preparation of a Standard Hematopoietic CFC Assay

Human BM, CB or MPB mononuclear cells were suspended in a methylcellulose-based culture medium containing cytokines (MethoCult™ H4434, StemCell Technologies) (StemCell Technologies Methocult™ Flyer; StemCell Technologies 2003 Catalogue pp. 86-125) at a concentration of between 1×10⁴ and 1×10⁵ cells per 1.1 mL of medium. The culture medium was then dispensed into 35 mm culture dishes using 3 mL syringes with blunt end needles, with 1.1 mL of medium per dish. All cultures were incubated for a total of 14 days at 37° C., 5% CO₂, 100% humidity. Colonies were enumerated and classified by bright-field microscopy into the following categories based on size and morphology: CFU-E, BFU-E, CFU-GM, CFU-GEMM (StemCell Technologies Colony Atlas).

### Example 2

### Staining of Erythroid Colonies With Fluorescent Antibody Conjugates

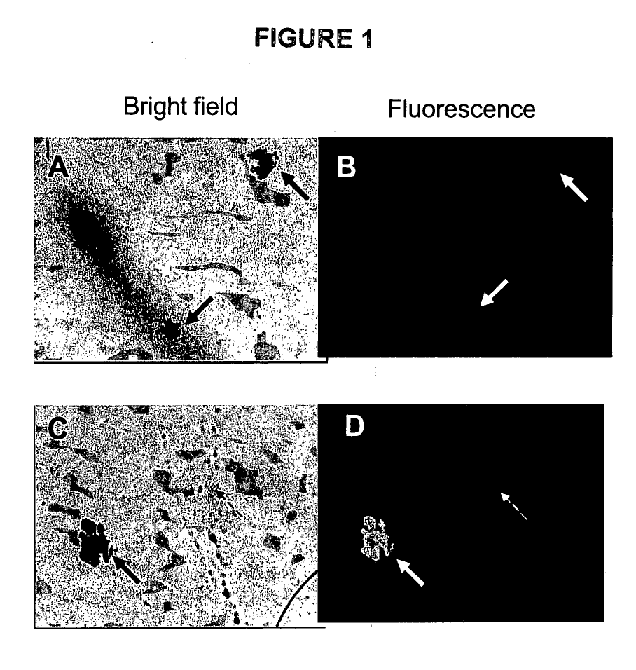

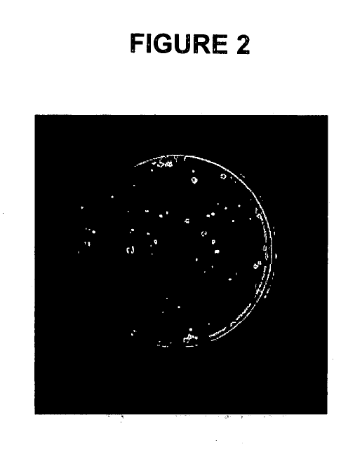

Standard CFC assays were prepared as described in example 1. At the time of plating, 1 to 10 μg of a FITC-conjugated antibody against Glycophorin-A (anti-GpA, catalogue number 10423, StemCell Technologies) was added per mL of medium. GpA is expressed on erythroid cells and is absent on other cell types, including myeloid cells. As a negative control separate dishes were stained in an identical manner with a FITC-conjugated control antibodies against a marker that is not expressed on eukaryotic cells (IgG2b-FITC, catalog number 557701, Becton-Dickinson, San Jose, Calif.). After 14 days of culture at 37° C. the dishes were inspected using an inverted fluorescence microscope. FIG. 1 shows photographs of section of dishes that were inspected at high magnification using either bright field illumination (FIGS. 1A and 1C) or ultra-violet (UV) illumination to detect fluorescence (FIGS. 1B and 1D). The photographs in FIG. 1A and B are for the same section of a dish stained with the negative control, FIG. 1C and D for the same section of a dish stained with anti-GpA-FITC. FIG. 1A shows the bright field images of two erythroid colonies (black arrows). FIG. 1B shows that these colonies were not fluorescent after staining with the negative control. FIG. 1C shows the bright field image of an erythroid colony (black arrow) and a myeloid colony (dashed arrow). FIG. 1D shows that the erythroid colony stained with the anti-GPA antibody was brightly fluorescent, while the non-erythroid colony in the same section of the dish was undetectable by UV illumination. FIG. 2 shows a composite fluorescent image of a whole dish stained with anti-GpA-FITC. Multiple brightly fluorescent colonies are visible. The erythroid colonies were counted using bright field and fluorescence. Both methods gave very similar results: 27 colonies per dish by bright field illumination; 26 colonies by fluorescence. The example shows that specific colony types can be specifically stained and identified without interference by other colony types, thereby increasing the ability to accurately identify and enumerate specific colony types.

### Example 3

### Monitoring of Erythroid Colony Development and Changes in Erythroid Antigen Expression During Culture.

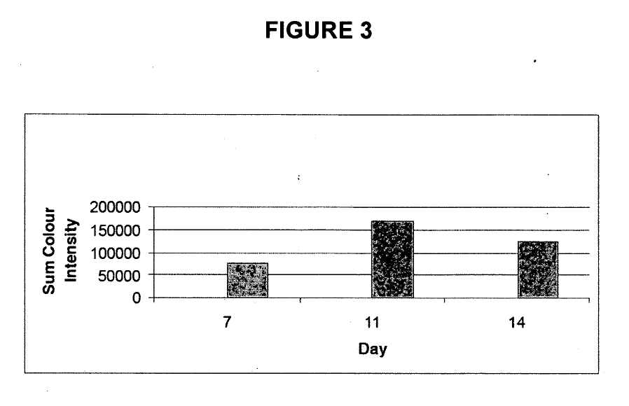

A CFC-assay was set up as described in Example 2. The culture dishes were inspected daily and the fluorescence of selected colonies were imaged at various time points. All microscope and image capture settings were kept constant. Colonies were analysed by densitometry using AnalySIS software (Soft Imaging System Corp., Riverside, Calif.) and fluorescence intensity was calculated for each time point. FIG. 3 shows that fluorescence of an anti-GPA-FITC stained erythroid colony was detectable as early as day 7, reached maximum intensity at day 11 and declined at the end of the culture period at day 14.

### Example 4

### Detection of Changes in Erythroid Colony Formation in Cultures Containing the Cytotoxic Compound 5-Fluoro-Uracil.

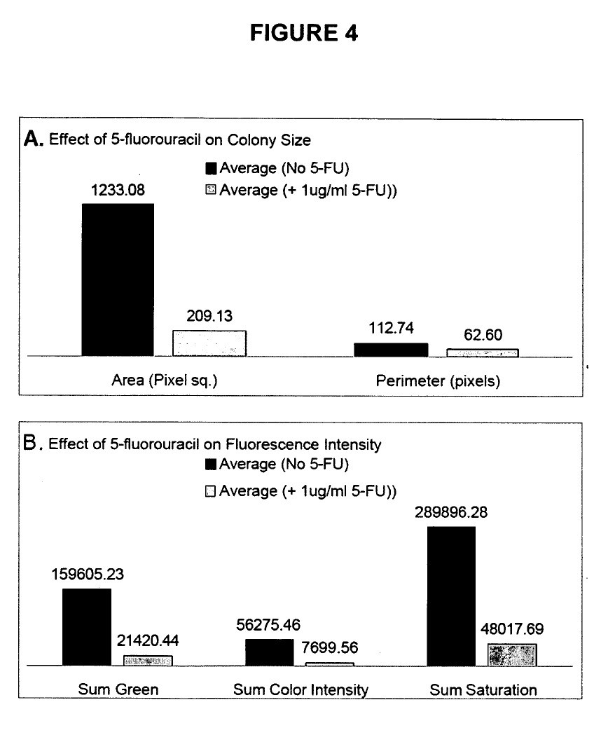

A CFC-assay was set up as described in Example 2. One set of culture dishes were supplemented with the cytotoxic compound 5-Fluoro-Uracil (5-FU). Colonies were examined and photographed on day 14 and colony size and fluorescence intensity were analysed by densitometry. As shown in FIG. 4A, the size of fluorescent colonies, as measured by surface area and perimeter length, was decreased in cultures containing 5-FU, which is consistent with an inhibitory effect of 5-FU on hematopoietic progenitor proliferation. As shown in FIG. 4B, fluorescence intensity was also decreased for colonies cultured in the presence of 5-FU, which indicated an inhibitory effect of 5-FU on erythroid differentiation. This example shows that effects of specific compounds on proliferation and differentiation of progenitor types can be detected and quantified by including fluorescently stained antibodies.

### Example 5

### Staining of CD45⁺ Colonies with Fluorescent Antibody Conjugates.

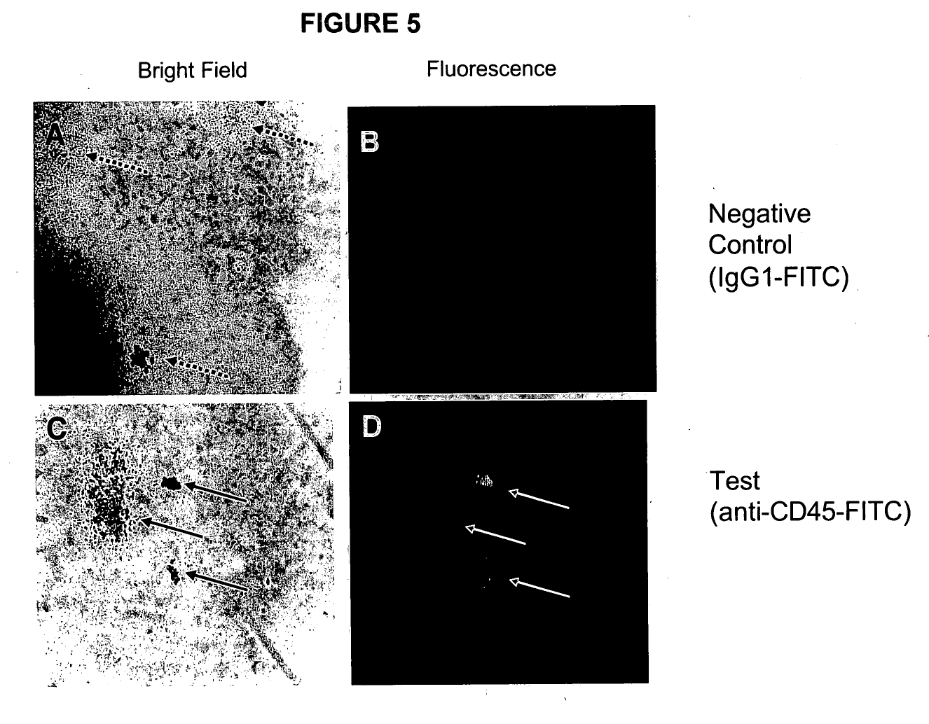

Standard CFC assays were prepared as described in Example 1. At the time of plating 5 μg of a FITC-conjugated antibody against human CD45 was added per mL of medium. CD45 is expressed on progenitors and on mature white blood cells, is more weakly expressed on differentiating erythroid precursor cells and is absent from mature red blood cells. As a negative control cells were also stained with a FITC-conjugated isotype control antibody. FIG. 5A and 5B show the bright field and fluorescence images of a section of the dish stained with the negative control. The location of three colonies, identified by bright field illumination are indicated by the arrows. A very faint background fluorescence is visible for some cells in the colony at the bottom, whereas both colonies at the top of the image are nonfluorescent (FIG. 5B). FIG. 5C and D show the bright field and fluorescence images of a section of a dish stained with anti-CD45-FITC. One colony with strong fluorescence and two with weaker fluorescence are indicated by the arrows. The brightly CD45⁺ colony indicated by the top arrow is an immature BFU-E, containing many immature CD45⁺ erythroblasts. The small dimmer colony (bottom) is a more mature BFU-E consisting of more differentiated erythroblasts that have partially lost CD45; The large CD45⁺ colony at the left is a myeloid colony containing many CD45⁺ myeloblasts.

These results show that immature and mature erythroid colonies in standard CFC assays can be identified on the basis of differences in staining intensity with anti-CD45-F while myeloid colonies can be identified on the basis of their morphology and expression of CD45.

### Example 6

### Staining of Mouse Granulocyte Colonies

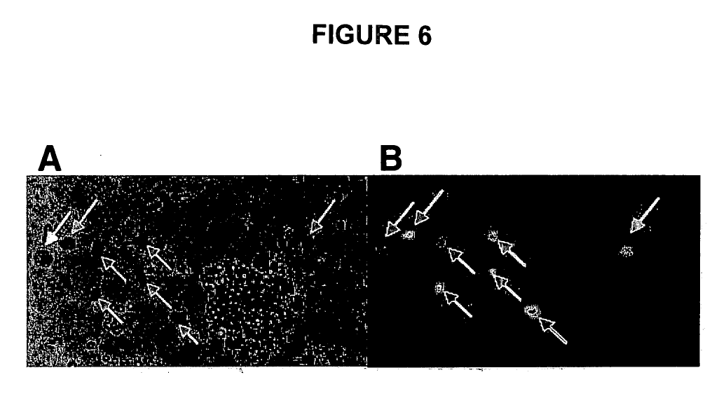

Mouse BM cells were suspended in a methylcellulose-based culture medium containing mouse cytokines (MethoCult™ M4434, StemCell Technologies) (StemCell Technologies Methocult™ Flyer; StemCell Technologies 2003 Catalogue pp. 86-125) at a concentration of between 1×10⁴ and 1×10⁵ cells per 1.1 mL of medium. The culture medium was then dispensed into 35 mm culture dishes using 3 mL syringes with blunt end needles, with 1.1 mL of medium per dish. All cultures were incubated for a total of 9 days at 37° C., 5% CO₂, 100% humidity. A rat monoclonal antibody against mouse Gr-1 conjugated to FITC was added to the culture dishes at a concentration of 10 μg/ml. Gr-1 is an antigen that is expressed on mouse granulocytes. Parallel cultures received a FITC-conjugated isotype control antibody at the same concentration, as a negative control. The culture dishes were incubated for 3 more days after which they were examined by bright field and fluorescent illumination. As shown in the bright field image in FIG. 6A several small colonies were visible as well as one large colony. The small colonies consisted of tight clusters of small cells. These colonies either represent granulocyte or erythroid colonies, which are difficult to distinguish accurately in mouse CFC assays. As shown in FIG. 6B only the small colonies were stained with the anti-Gr-1-FITC antibody, indicating that these were granulocyte colonies. The large colony remained non-fluorescent indicating that this was not a granulocyte colony. The morphology of this colony was consistent with that of monocyte/macrophage colonies, which do not express Gr-1. This example showed that specific mouse colonies can also be identified by staining with fluorescent antibodies against specific antigens during culture and that Gr-1 expression can be used to differentiate between granulocyte and monocyte/macrophage colonies.

### Example 7

### Preparation of a Standard Neural CFC Assay

Neural cells can be obtained from primary embryonic, post-natal or adult CNS tissue from any region of the neuroaxis including but not limited to the striatum, septum, cortex, ventral mesencephalon, septum, midbrain, cerebellum or spinal cord from murine, rodent and human. Neural cells can also be obtained from cultured cells such as those generated using the Neurosphere Assay or any method known to one skilled in the art of neural tissue culture. Neural cells can also be obtained from any stage of embryonic stem cell cultures according to any standard procedure for culturing ES cells.

For example, striata and/or cortex are dissected from Embryonic Day 14 CD₁ albino mouse embryos (Charles River) using standard microdissection techniques. Tissue is collected in phosphate-buffered saline with 2% glucose then mechanically dissociated using a fire-polished glass pipette into a single cell suspension, washed once and filtered through a 40 μm nylon cell strainer (Falcon) and diluted to a concentration of 2.17×10⁵ cells per/mL in complete NeuroCult™ medium (NeuroCult™ Basal Medium & NeuroCult™ Proliferation Supplements; StemCell Technologies Inc. with 20 ng/ml of EGF).

Alternately, cells from cultured neurospheres such as those derived from tumor tissues, such as glioblastoma multiforme (GBM), can also be used for the NCFC assay. Tissue is dissected using standard microdissection techniques. Tissue is collected in phosphate-buffered saline with 2% glucose then dissociated into a single cell suspension and plated in a complete NeuroCult™ medium (NeuroCult™) Basal Medium & NeuroCult™ Proliferation Supplements; StemCell Technologies Inc.) with 20 ng/ml of EGF (10 ng/mL of bFGF and 0.2 ug/mL of heparin are included in the human cell cultures). Cells are cultured for 7 days to generate neurospheres for use in the N-CFC assay. Day 7 neurospheres are collected from the culture, mechanically dissociated into a single cell suspension, filtered through a 40 μm nylon cell strainer (Falcon).

A single cell suspension of neural cells produced from either of the two examples mentioned above are diluted to a concentration of 2.2×10⁵ cells per/ml in complete NeuroCult™ medium (StemCell Technologies Inc.). To make a 3.3 ml solution of the semi-solid NSC assay media add the following components in the given order:

| | | |
|---|---|---|
| NeuroCult ® N-CFC serum-free medium without | 1700 | ul |
| cytokines (StemCell Technologies) | | |
| NeuroCult ® Proliferation Supplements | 330 | ul |
| (StemCell Technologies) | | |
| Epidermal Growth Factor (10 mg/ml) | 6.6 | ul |
| Cells (2.2 × 10⁵ cell/ml) | 25 | ul |
| Collagen (Bovine, StemCell Technologies) | 1300 | ul |
| Total Volume | 3361 | ul |

Mix the resulting solution well to evenly distribute the cells throughout the medium. 1.5 ml of the suspension is plated into individual 35 mm tissue culture plates at a final density of 2500 cells per dish. Cultures are placed in a tissue culture incubator set at 37° C., 100% humidity and 5% CO₂. The colonies are enumerated and sized number between day 14-28.

Within 4-7 days of plating in the N-CFC assay NSC and neural progenitor cells begin to proliferate forming small colonies. By 14 days these small colonies have grown in size and differences can be discerned between colonies. A number of the colonies appear to stop growing after approximately 10-14 days while other colonies continue to expand. By 21-28 days, colonies can be classified into at least 4 categories: 1) greater than 2 mm in diameter, 2) 1-2 mm in diameter, 3) 0.5 - 1 mm in diameter and 4) less than 0.5 mm in diameter. Colonies can be sized and counted and the frequency of colonies within each of these size categories graphed

### Example 8

### Staining of Neural Colonies in a Neural CFC Assay.

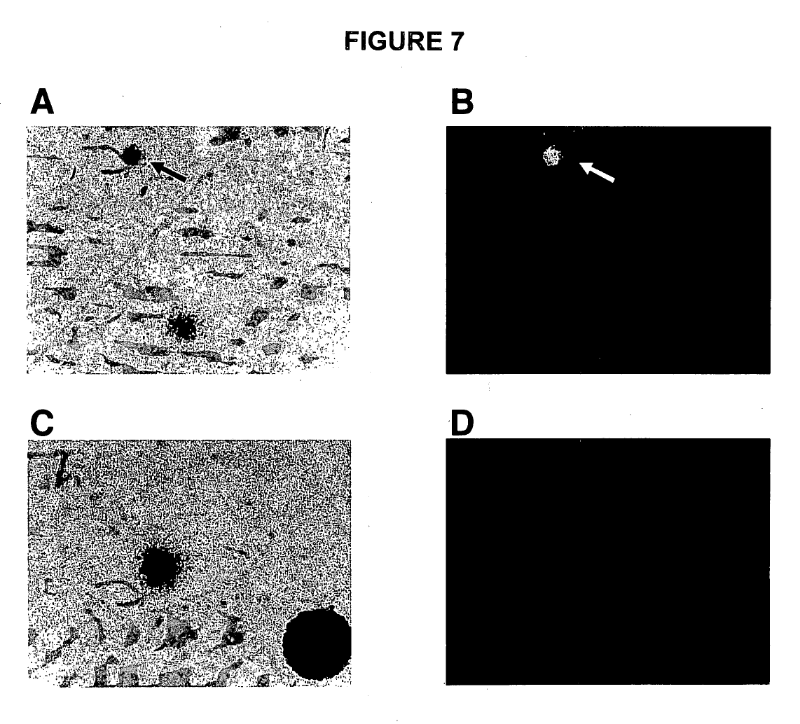

A NCFC-assay was set up using human glioblastoma multiforma cells as described in Example 7. After 21 days in culture anti-CD15-FITC (BD-Biosciences, catalog # 347428) was added to a final concentration of 10 μg/ml, while parallel cultures received a control antibody (anti-KLH-FITC; BD-Biosciences, catalog # 349041) at the same concentration. The cultures were continued for another 1-2 days, after which the cultures were inspected under an inverted microscope using bright field and UV illumination. Bright field and fluorescence images were recorded for representative sections of each dish. For fluorescence microscopy the exposure time for the test and control dishes was set at 700 ms to enable direct comparison between both sets of dishes. As shown in FIG. 7 fluorescent colonies were detected in anti-CD15-FITC stained dishes (A,B), while all colonies in the control dish were non-fluorescent (C,D). As summarized in Table 1, 8 out of a total of 49 NCFC colonies in the dish shown in FIGS. 7A and B were positive, compared to 0 out of 34 colonies in the control dish in FIGS. 7C and D.

TABLE 1
CD15 expression on human NCFC colonies

| | | CD15-FITC stained | | IgG1-FITC (negative | |
|---|---|---|---|---|---|
| | | colonies | | control) colonies | |
| | | Total # of | CD15-FITC | Total # of | IgG1-FITC |
| Colony Size | | Colonies | Positive | Colonies | Positive |
| <1 | mm | 34 | 8 | 24 | 0 |
| 1-2 | mm | 12 | 0 | 8 | 0 |
| >2 | mm | 3 | 0 | 2 | 0 |

### Example 9

### Modified CFC-Assay for Erythroid Colonies.

As a modification of the standard CFC assay peripheral blood mononuclear cells were suspended in a serum free methylcellulose-based culture medium (MethoCult™, H4236, StemCell Technologies), supplemented with SCF (100 ng/ml) and EPO (3 Units/ mL). The culture media were dispensed and the cultures incubated, enumerated and classified as described in Example 1. In contrast to a standard CFC assay only erythroid colonies (BFU-E) developed in these cultures. The erythroid colonies had similar size and morphology as erythroid colonies grown in standard CFC assays, and were identified at slightly lower frequencies: 29+/−14 (average +/−standard deviation) colonies per 10⁵ blood cells in the standard CFC-assay compared to 25+/−16 colonies in the modified CFC-assay, but BFU-E frequencies correlated well between both assays (p<0.1, two-tailed t-test, n=18). Other colony types (CFU-GM, CFU-GEMM) were much smaller than the erythroid colonies and present in much lower frequencies than the same types of colonies grown in a standard CFC-assay. The selective growth of erythroid colonies reduced variability in enumeration and classification of erythroid colonies and reduced the time required to enumerate these colonies.

### Example 10

### Enrichment of Colony-Forming Cells

Cells expressing CD34 were isolated using the EasySep® immunomagnetic separation method (StemCell Technologies). Non-purified and purified CD34⁺ cells were stained with a FITC-conjugated anti-CD34 antibody and CD34⁺ cells in each cell preparation were detected and enumerated by flow cytometry. CD34⁺ cells were enriched 50-100-fold in the purified CD34⁺ cell preparation relative to the non-purified cell preparation. Preparations of non-purified cells (1×10⁴-2×10⁵ cells per culture) and of purified CD34⁺ cells (500-2000 cells per culture) were then cultured, enumerated and classified as described in Example 1. The types and number of colonies in each culture was similar, but in the cultures of purified CD34⁺ cells individual colonies were more easily distinguished from each other, because the background due to mature cells and non-CFCs was much lower than in the cultures of non purified cells. This example shows that enrichment of CFCs improves the contrast between colonies and background and between colony types, reduces variability by introducing a more uniform population of cells and reduces the time required to detect and enumerate individual colonies.

### Example 11

### Reduction of Background Erythroid Colony Formation by Purified CD34⁺ Cells

Cells expressing CD34 were purified as described in Example 10. Samples of non-purified cells and purified CD34⁺ cells were then cultured in a modified methylcellulose-based medium containing SCF and EPO, optimized for erythroid colony growth, as described in Example 2. In addition, samples of the same cell preparations were cultured in a modified medium, as described in Example 2, except that EPO was not added to the culture medium. After culture colonies were enumerated and classified as described in Example 1. Only erythroid colonies were detected in the cultures of non-purified cells and of purified CD34⁺ cells in culture media containing SCF and EPO. Erythroid colonies, identified on the basis of their morphology and red colour due to hemoglobinization, were also detected in cultures of non-purified cells in culture medium containing SCF, but no added EPO. These are background erythroid colonies, i.e., colonies that grow in cultures that have not been supplemented with EPO. The frequency of these background erythroid colonies was approximately one third (mean +/− standard deviation=28%±9.6%, 12 experiments) of the frequency of erythroid colonies that grew in medium containing SCF and EPO, and their size was much smaller. The frequency of background erythroid colonies in cultures of purified CD34⁺ cells grown in medium supplemented with SCF, but not EPO was only 2 (range 0-3%). This example shows that background erythroid colony formation in the absence of added EPO can be greatly reduced by using a modified culture system using purified CD34⁺ cells instead of non-purified cells, in combination with a modified serum-free culture medium.

### Example 12

### Reduction of Background Erythroid Colony Formation by Cell Preparations Depleted of Monocytes.

Blood cells were stained with antibodies against CD14, which is a marker for mature monocytes. The CD14⁺ monocytes were then depleted by immunorosetting using the RosetteSep® methodology (StemCell Technologies). Erythroid colonies in the total and monocyte depleted cell preparations were then cultured, enumerated and classified in modified colony assays as described in Example 9. The frequency of background erythroid colonies in cultures supplemented with SCF was only 2.6%±1.3% (mean±standard deviation, 4 experiments) in the monocyte depleted cell preparations, compared to 28%±9.6% (12 experiments) for blood cell preparations from which monocytes had not been depleted. This example shows that background erythroid colony formation in the cultures not supplemented with EPO can be reduced by using a modified culture system using cell preparations from which specific subsets of cells, in this example monocytes, have been depleted, in combination with a modified serum-free culture medium.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Bacigalupo A, Piaggio G, Podesta M et al. Influence of marrow CFU-GM content on engraftment and survival after allogeneic bone marrow transplantation. Bone Marrow Transplantation: 15, 221-226, 1995.
2. Torres A, Alonso M. C., Gomez-Villagran J. L. et al. No influence of number of donor CFU-GM on granulocyte recovery in bone marrow transplantation for acute leukemia. Blut 50: 89-94, 1985.
3. Burger S R, Kadidlo D and McCullough J. Improved progenitor assay standardization using peripheral blood mononuclear cells from a donor treated with granulocyte-colony stimulating factor. Transfusion 39: 451-456, 1999.
4. Lumley M A, Burgess R, Billingham L J et al. Colony counting is a major source of variation in CFU-GM results between centres. British Journal of Haematology 97: 481-484, 1997.
5. Lamana M, Albella B, Rodriguez F et al. Conclusion of a national multicenter incomparative study of in vitro cultures of human hematopoietic progenitors. Bone Marrow Transplantation 23: 373-380, 1999.
6. Colony Atlas. StemCell Technologies Inc, Vancouver, Canada. Catalog number 28700.
7. StemCell Technologies Methocult™ Flyer, http://www.stemcell.com/technical/methocult01.pdf.
8. StemCell Technologies 2003 Catalogue pp. 86-125.
9. Reynolds B A, Tetzlaff W, Weiss S. A multipotent EGF-responsive striatal embryonic progenitor cell produces neurons and astrocytes.J Neurosci. 12:4565-74, 1992.
10. Reynolds B A, Wagey R, Thomas T E, Eaves A C, Louis S A. An in vitro colony assay which identifies neural stem cells with high and low proliferative potential [abstract]. 2003 Abstract Viewer/Itinerary Planner. Washington, D.C.: Society for Neuroscience, 2003.Online 2003;Program No. 124.5.
11. Stingl J, Eaves C. J, Kuusk U, Emerman J. T. Phenotypic and functional characterization in vitro of a multipotent epithelial cell present in the normal adult human breast. Differentiation 63:201-213, 1998.
12. Stingl J, Eaves C J, Zandieh I, Emerman J T Characterization of bipotent mammary epithelial progenitor cells in normal adult human breast tissue.

Breast Cancer Res Treat. 67: 93-100, 2001.
13. Pechoux C, Gudjonsson T, Ronnov-Jessen L, Bissell M J, Petersen O W.

Human mammary luminal epithelial cells contain progenitors to myoepithelial cells. Dev Biol. 206: 88-99, 1999.
14. Smalley M J, Titley J, O'Hare M J. Clonal characterization of mouse mammary luminal epithelial and myoepithelial cells separated by fluorescence-activated cell sorting. In Vitro Cell Dev Biol Anim. 34: 711-721, 1998.
15. Smalley M J, Titley J, Paterson H, Perusinghe N, Clarke C, O'Hare M J. Differentiation of separated mouse mammary luminal epithelial and myoepithelial cells cultured on EHS matrix analyzed by indirect immunofluorescence of cytoskeletal antigens. J Histochem Cytochem. 47:1513-1524,1999.

1. A method of detecting progenitor cells in a starting cell preparation comprising:
(a) culturing the cell preparation in a culture medium suitable to promote the growth and differentiation of the progenitor cells into colonies containing a specific cell type;
(b) adding a detection reagent that can detect the progenitor cells or the specific cell type; and
(c) detecting progenitor cells or the specific cell type wherein the presence of such cells indicates the presence of progenitor cells in the starting cell preparation.

2. The method according to claim 1 wherein the detection reagent is added to the culture vessel at the beginning of the culture or during culture.

3. The method according to claim 1 wherein development of specific cell types is detected during culture by monitoring changes in reactivity with the detection reagent and without destruction of the culture.

4. The method according to claim 1 wherein the detection reagent is an antibody.

5. The method according to claim 4 wherein the antibody is labeled.

6. The method according to claim 5 wherein the antibody is labelled with a fluorescent marker.

7. The method according to claim 1 wherein the specific cell type is a hematopoietic cell.

8. The method according to claim 1 wherein the detection reagent is an antibody specific for erythroid cells.

9. The method according to claim 8 wherein the antibody is specific for glycophorin A.

10. The method according to claim 1 wherein the detection reagent is an antibody specific for non-erythroid cells.

11. The method according to claim 10 wherein the antibody is specific for CD45.

12. The method according to claim 1 wherein the detection reagent is an antibody specific for myeloid cells.

13. The method according to claim 12 wherein the antibody is specific for CD11 b, CD14, CD15, CD66b or Ly-6G (Gr-1).

14. The method according to claim 1 wherein the detection reagent is an antibody specific for megakaryocytic cells.

15. The method according to claim 14 wherein the antibody is specific for CD41a.

16. The method according to claim 1 wherein the detection reagent is an antibody specific for HSCs and progenitors.

17. The method according to claim 16 wherein the antibody is specific for CD34.

18. The method according to claim 1 wherein the culture medium contains stem cell factor (SCF).

19. The method according to claim 18 to detect erythroid cells wherein the culture medium also contains erythropoietin (EPO).

20. The method according to claim 18 wherein the culture medium does not contain EPO.

21. The method according to claim 19 wherein the culture medium is serum-free.

22. The method according to claim 18 to detect myeloid cells wherein the culture medium also contains GM-CSF, IL-3, IL-6 and/or G-CSF.

23. The method according to claim 1 wherein the starting cell preparation is depleted of a population of cells that are not the specific cell type prior to culturing the cells in step (a).

24. The method according to claim 23 wherein the cells are depleted of monocytes.

25. The method according to claim 1 wherein the specific cell type is a neural cell.

26. The method according to claim 1 wherein the detection reagent is an antibody specific for neurons.

27. The method according to claim 26 wherein the antibody is specific for Neurofilaments NF-H, NF-L or NFL-M or for Class II beta-Tubulin.

28. The method according to claim 1 wherein the detection reagent is an antibody specific for astrocytes.

29. The method according to claim 28 wherein the antibody is specific for Glial Fibrillary Acidic Protein (GFAP).

30. The method according to claim 1 wherein the detection reagent is an antibody specific for oligodendrocytes.

31. The method according to claim 23 wherein the antibody is specific for Myelin Basic Protein (MBP).

32. The method according to claim 1 wherein the detection reagent is an antibody specific for neural stem cells.

33. The method according to claim 32 wherein the antibody is specific for CD15.

34. The method according to claim 1 wherein the specific cell type is a mammary cell.

35. The method according to claim 1 wherein the detection reagent is an antibody specific for mammary stem cells, myoepithelial cells or luminal epithelial cells.

36. The method according to claim 35 wherein the detection reagent is an antibody specific for CD24, Keratin-18, Keratin-19, EpCAM, MUC-1 or CD10.

37. A method of detecting progenitor cells to a specific cell type in a starting cell preparation comprising:
(a) depleting a population of cells that are not the specific cell type in the cell preparation;
(b) culturing the depleted cell population in a culture medium suitable to promote the growth and differentiation of the progenitor cells into colonies containing the specific cell type; and
(c) detecting the progenitor cells or the specific cell type in the cell population.

38. The method according to claim 37 wherein monocytes are depleted in step (a).

39. The method according to claim 37 wherein the culture medium is serum free and contains SCF.

40. The method according to claim 37 wherein the culture medium does not contain EPO.

41. The method according to claim 37 wherein the monocytes are removed by negative selection.
